(19) United States
(12) Patent Application Publication
(10) Pub. No.: US 2009/0259109 A1
(43) Pub. Date: Oct. 15, 2009

(54) Sternum retractor device

(75) Inventors: Massimo Bucefari, Arezzo (IT); Francesco Santini, Parona (Verona) (IT)

Correspondence Address: POLLACK, P.C., THE CHRYSLER BUILDING, 132 EAST 43RD STREET, SUITE 760, NEW YORK, NY, 10017, US

(73) Assignee: Elenor S.r.l.

(21) Appl. No.: 12/321,342

(22) Filed: Jan. 19, 2009

(30) Foreign Application Priority Data
Jan. 18, 2008 (IT) FI2008A000007

(51) Int. Cl. A61B 1/32

(52) U.S. Cl. 600/219

(57) ABSTRACT

A sternum retractor device is disclosed suitable for temporarily maintaining a selected gap between two sternal stumps during cardiac surgical procedures. The device comprises a pair of jaws configured so as to be inserted between the stumps and shaped for firmly engaging the stumps, an actuator connected to the jaws for controlling their mutual closing and opening movements, and a member for controlled feed or withdrawal of a selected biocompatible actuator fluid in/from the actuator, thereby enabling the achievement and maintenance of the gap required between the stumps as well as the progressive reduction of the gap as a function of a patient's condition.

### FIELD OF THE INVENTION

The present invention relates generally to surgical devices and, more particularly, to a device for use in cardiac surgical procedures or the like.

### BACKGROUND OF THE INVENTION

During the final stages of cardiac surgical procedures, there may sometimes be prolonged periods of left and/or right ventricular dysfunction resulting from preoperative clinical conditions (e.g., ventricular function, bronchopneumonia, renal dysfunction) and/or lesions corresponding to the procedure underway (such as myocardial protection modalities, extracorporeal circulation, and/or intraoperative injuries). These dysfunctions are often refractory to common resuscitation techniques (catecholamine infusion, diuretics, intra-aortic counterpulsation, and the like).

In such circumstances, it may prove unfeasible to close the sternum, especially when the cardiac dysfunction is associated with severe myocardial, pulmonary or mediastinal tissue oedema. This is because the extrinsic compression exerted by the sternum further worsens the already strained cardiopulmonary function of the engorged and congested mediastinal organs, with potentially severe haemodynamic and respiratory consequences, that are sometimes even incompatible with survival.

In the above circumstances, postponing the closure of the sternum is now considered an accredited and potentially indispensable manoeuvre, both in routine clinical experience and in the literature.

Other indications for delaying the closure of the sternum include refractory mediastinal bleeding, malignant ventricular arrhythmias correlated with myocardial oedema, and external pulmonary emphysema with potentially severe extrinsic compression even on a normally-functioning heart.

Postponing the closure of the sternum does not simply consist in leaving the sternum open. Given the above-mentioned premises, the sternal stumps (after longitudinal incision of the sternum) need to be kept spaced apart not only to reduce their extrinsic compression on the mediastinal structures as far as possible, but also to avoid the sharp profiles of the bone stumps coming into contact with the heart (right ventricle) and lung and causing traumatic lesions.

The amplitude of the artificially-induced gap between the two sternal stumps may need to be varied to suit the circumstances, but at the beginning of the procedure it is generally between 8 and 10 cm for an adult individual of average size.

In the past, endotracheal tubes, or the tubes of the extracorporeal circulatory circuit used for the patient during the surgical procedure, or plastic syringes, etc, have been used to establish and maintain this artificial divarication by inserting them between the two sternal stumps to keep the thoracic cavity open. Once the retractor device had been positioned, the surgical site was covered with a synthetic membrane stitched along its edges to prevent infections and contaminations.

These makeshift and sometimes rudimental retractor devices have often given rise to complications correlated with their stability, dimensions and the sturdiness of the materials used. For instance, a syringe with its edges cut to the required shape may become sharp and, as a consequence, potentially traumatic for the surface of the right ventricle lying underneath. In fact, traumatic lesions of the anterior surface of the right ventricle correlated with this risk have been reported.

In addition to the inadequacy of the materials, the devices used in the past to artificially divaricate the sternal stumps were simply inserted between the stumps without the opportunity to adjust the distance between them. As is self-evident to a person skilled in the art, there may be two reasons for needing to adjust this distance:

1. if the patient's haemodynamic conditions become worse, it may be necessary to further widen the gap between the sternal stumps, thereby further reducing the pressure on the mediastinal organs;

2. if the patient's haemodynamic conditions improve, it may be useful to gradually reduce the distance between the stumps, also with a view to testing the patient's tolerance of any final closing manoeuvre.

With the methods used to date, both such situations demanded the reopening of the surgical lesion (with the related reiterated risk of infection), removing the object inserted as a retractor (a syringe or other object) and replacing it with another, shorter or longer object, according to need.

Although they are considered “lifesavers”, none of the retractor systems used to date have ever been validated by the competent authorities (FDA, CE, etc.).

### OBJECTS AND SUMMARY OF THE INVENTION

Accordingly, it is an object of the present invention to provide a temporary sternum retractor device for use in postponing closure of the sternum after a cardiac surgical procedure, that enables problems concerning maintaining and controlling the extent of sternum divarication to be safely and efficiently overcome, while avoiding risks associated with conventional makeshift devices.

A particular object of the present invention is to provide a retractor device of the above-mentioned type that can be firmly attached to the sternal stumps, which respond to the ventilator-induced respiratory movements, so that it cannot be displaced and consequently damage the mediastinal organs.

A further object of the present invention is to provide a retractor device of the above-mentioned type that can be configured to have a low profile so that it does not extend beyond the outer edges of the skin incision in order to allow for the lesion to be closed with a synthetic membrane to maintain sterile conditions at the surgical site.

These advantages are achieved by the temporary sternum retractor according to the present invention, the essential characteristics of which are stated in claim 1.

### BRIEF DESCRIPTION OF THE DRAWINGS

A specific, illustrative sternum retractor device, according to the present invention, is described below with reference to the accompanying drawings, in which:

FIG. 1 is a perspective view of a sternum retractor device in a closed position, according to one aspect of the present invention;

FIG. 2 shows the device of FIG. 1 in a fully-open position;

FIG. 3 is a side view of the device taken along arrow F in FIG. 2;

FIG. 4 is an opposite view of the device as illustrated in FIG. 2;

FIG. 5 is a perspective view of a retractor device in a closed position, according to another aspect of the present invention;

FIG. 6 shows the device of FIG. 5 in a fully-open position;

FIG. 7 is a sectional view of the device illustrated in FIG. 5; and

FIG. 8 is a sectional view of the device set forth in FIG. 6.

The same numerals are used throughout the drawing figures to designate similar elements. Still other objects and advantages of the present invention will become apparent from the following description of the preferred embodiments.

### DETAILED DESCRIPTION OF THE PREFERRED EMBODIMENTS

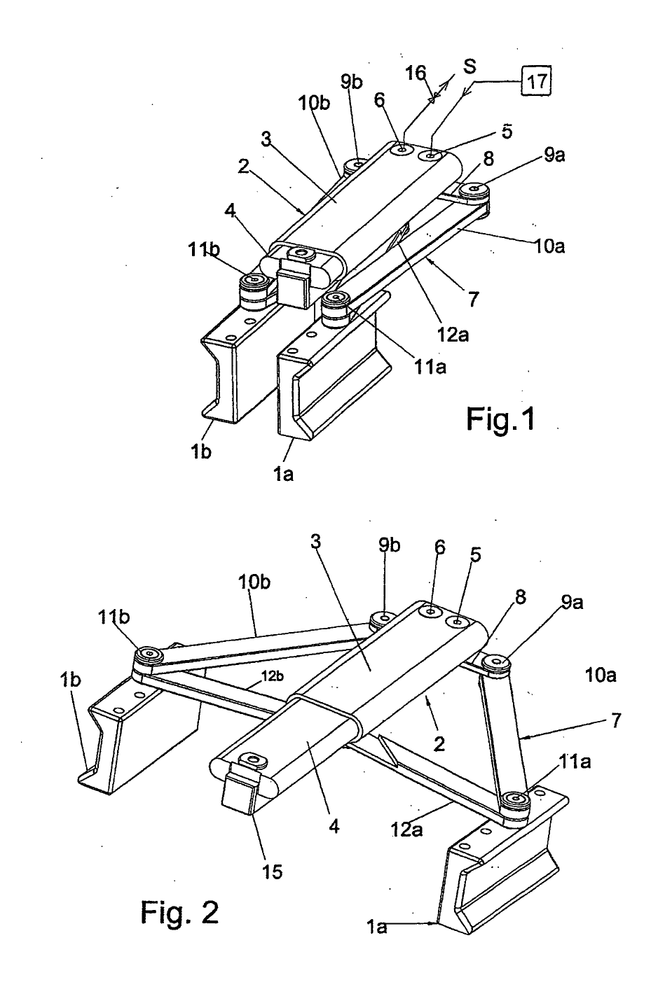

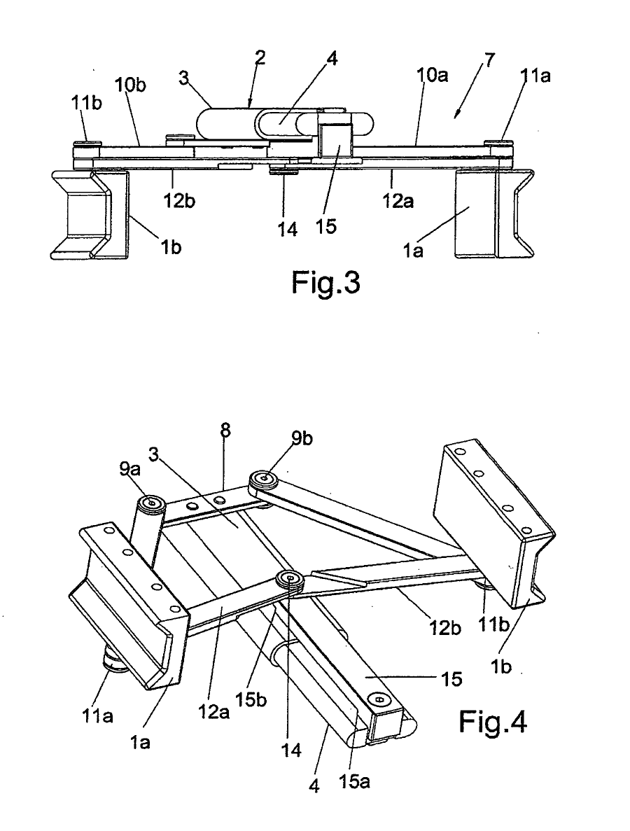

Referring now to the drawings and, more particularly, to FIGS. 1-8, there is shown generally a specific, illustrative sternum retractor device, according to various aspects of the present invention. In one embodiment, illustrated in FIG. 1, the device comprises a plurality, e.g., a pair, of relatively parallel, opposing jaws 1a, 1b, with a substantially U-shaped cross-section so as to house two sternal stumps and provide an abutment surface on which the force needed to keep them spread apart is applied. Jaws 1a, 1b are attached to an actuator device 2 which comprises a stationary part in the form of a tubular sleeve 3, closed at one end and having a very flat oval cross-section, in which a movable part in the form of a piston 4 is slidingly engaged in a watertight manner. An actuator fluid S is inserted in or extracted from the chamber inside sleeve 3, delimited by piston 4, through respective inlet and outlet ports 5 and 6 provided on the sleeve.

The tubular sleeve 3 lies parallel to the two jaws 1a, 1b and the actuator device 2 is connected thereto by means of an articulated mechanism generically indicated by the numeral 7. The articulated mechanism 7 comprises a bar 8 fixed crosswise to the tubular sleeve 3 and substantially at the end of said sleeve opposite from the end in which the piston 4 slides. The bar 8 extends laterally from both sides of the sleeve 3 and its ends are pivotally connected through pins 9a, 9b to the ends of respective first arms 10a, 10b. The first arms 10a, 10b are in turn revolvingly connected at their opposite ends with the respective jaws 1a, 1b through pins 11a, 11b integral to these jaws. Therefore, the first arms 10a, 10b extend along the sides of the tubular sleeve when the retractor device according to the invention is in its closed condition, as shown in FIG. 1.

The articulated mechanism 7 also comprises second arms 12a, 12b, with ends pivotally engaging with the pins 11a, 11b, while their opposite ends both engage with the same pin 13, shown in FIG. 4, integrally attached to the piston 4. More precisely, with reference to FIG. 4, the pin 14 projects perpendicularly from a bracket 15 extending along the piston 4 and at a distance therefrom that is greater than the thickness of the sleeve 3, so as to enable the displacement of the sleeve 3 between the piston 4 and the bracket 15. The end of the bracket 15 is attached to the free end of the piston 4 and, on the side facing towards the piston, it is formed with two shoulders indicated as 15a and 15b, with which it abuts against the free edge of the sleeve 3 and the crosswise bar 8, respectively, when the retractor device is in its closed position.

The axes of the pins 9a,b, 11a,b and 14 lie parallel to one another.

When the actuator fluid is fed to the sleeve 3, the piston 4 is displaced axially to emerge from the sleeve, causing the jaws 1a, 1b to move away in a direction orthogonal to the direction of said axial displacement of the piston 4 due to the effect of the articulated mechanism 7. In fact, the displacement of the piston has the effect of making the pin 14 slide axially too, extending both the second pair of arms 12a,b relative to the piston 4, and the first pair of arms 10a,b relative to the tubular sleeve 3, up to a maximally divaricated position substantially corresponding to the one shown in FIGS. 2, 3 and 4.

Extracting the actuator fluid from the sleeve 3 causes the piston 4 to withdraw progressively in the sleeve 3 and, as a consequence, the two jaws 2a,b to get close to one another until they return to the closed position shown in FIG. 1.

In use, the sternum retractor device according to the present invention is attached to the two sternal stumps, which engage in the jaws 1a,b, then actuator fluid is fed and the device is divaricated until it reaches the position best suited to the specific needs of the case. Given the kinematic configuration adopted, the opening force increases rapidly with the opening of the mechanism: in fact, a progressively greater force needs to be exerted on the two sternal stumps to open the rib cage. As the patient's conditions improve, the divarication of the device can be progressively reduced by withdrawing some of the actuator fluid from the sleeve 3.

The actuator fluid can advantageously consist of a biocompatible liquid, e.g. a physiological saline solution of known type compatible with this particular usage. The biocompatible liquid can be fed manually in the sleeve 3, by means of a syringe for instance, generally shown in FIG. 1 and indicated as 17, and withdrawn by opening a shut-off valve 16 located at the outlet from the sleeve 3 and exploiting the force exerted by the patient's rib cage which tend to close the opening in the sternum.

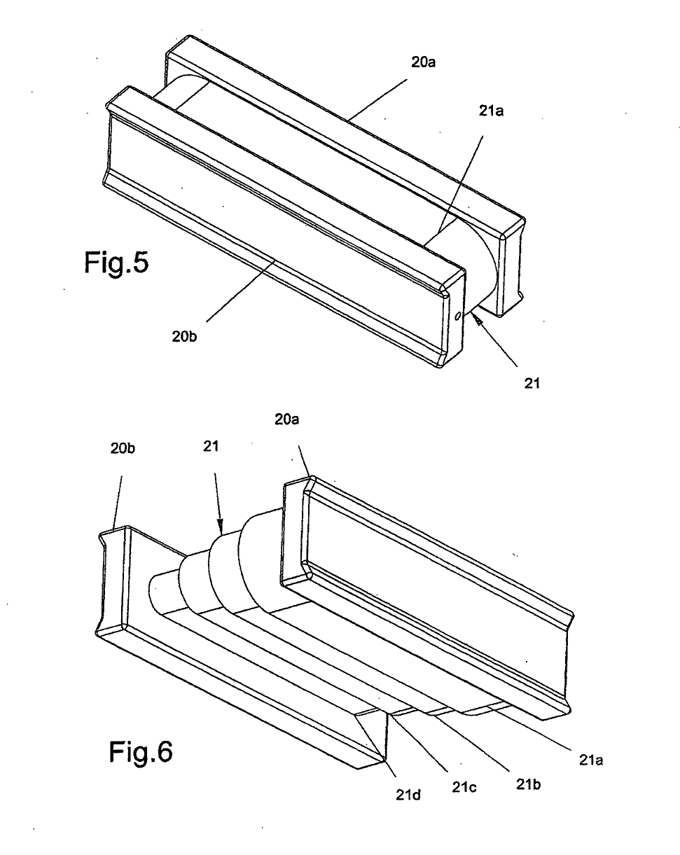

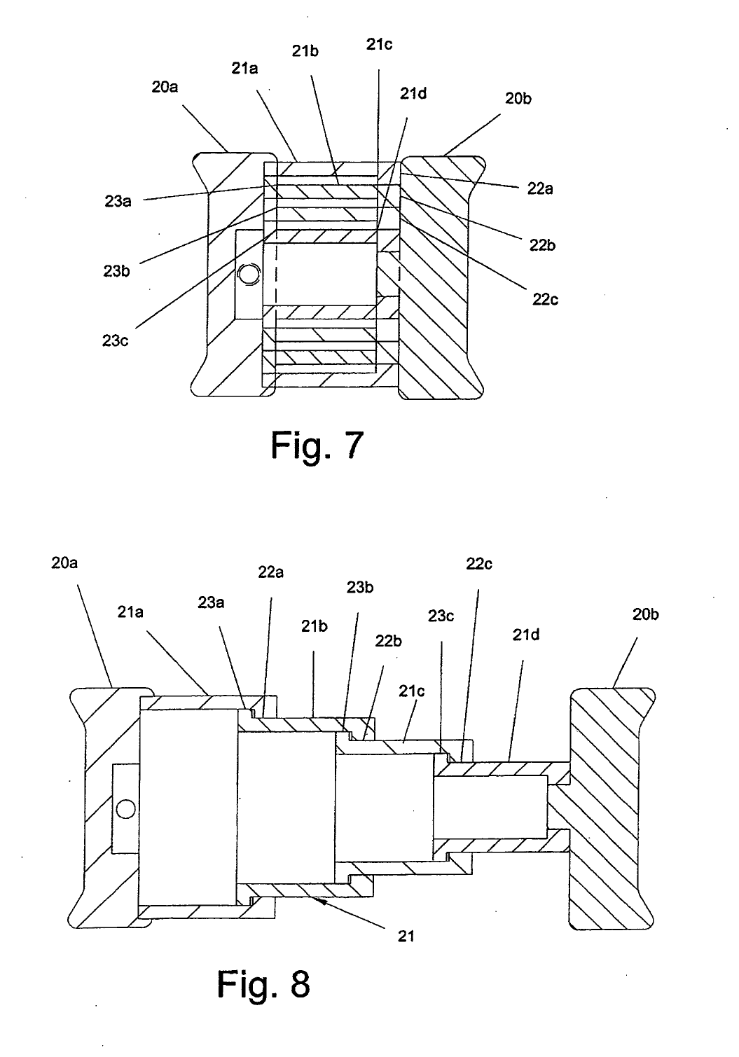

In a second embodiment of the invention, illustrated in FIGS. 5-8, the two jaws 20a, 20b are connected on opposite sides to a telescopic actuator device, generically indicated by the numeral 21. The telescopic actuator device 21 is formed by a plurality (four in the embodiment shown, identified as 21a, b, c, d) of progressively smaller-sized sleeves such that they can be contained one inside the other. As illustrated more clearly in the cross-sections in FIGS. 7 and 8, where the retractor device according to the invention is shown both in its closed condition (FIG. 7) and in its opened condition (FIG. 8), the widest terminal sleeve 21a is rigidly connected to one of the two jaws (e.g. the jaw 20a), while the opposite, narrowest terminal sleeve (21d) is rigidly connected to the other jaw (e.g. the jaw 20b). The sleeves 21a, b, c, d are slidingly connected to one another with a watertight connection, since the wider sleeves contain an internal flange (22a, b, c) that slides to form a watertight seal against the outer surface of the narrower sleeves, which in turn have an external flange 23a, b, c that slides to form a watertight seal against the inner surface of the wider sleeves. Here again, the mutual sliding between the sleeves 21a,d is achieved by feeding/withdrawing an actuator fluid, such as a biocompatible liquid, in/from the internal chamber delimited by the sleeves 21a,d through inlet/outlet ports.

The insertion of actuator fluid in the sleeves 21a,d causes the divarication of the jaws 20a,b and the consequent moving away of the sternal stumps from one another, while the withdrawal of actuator fluid from said chamber enables the two jaws 20a,b to move closer together, due to the effect of the force exerted by the rib cage. Here again, the actuator fluid can be inserted manually, e.g. by means of a syringe, up until a maximum divarication has been reached corresponding to the maximum extension of each sleeve from the wider sleeve containing it, and then releasing the volume of fluid needed to achieve the degree of divarication required case by case.

It will be clear to a person skilled in the art that, in the two above-described embodiments of the invention, as an alternative to said manual methods, there may be automatic systems for feeding and withdrawing the actuator fluid, such as a metering pump on a feed line and a solenoid valve on a discharge line, the operation of which is governed by a microcontroller.

It should be noted that, in both the embodiments, the actuator has a very flat oval cross-section in order to restrict its overall dimensions in the direction orthogonal to the sternal plane.

Since the retractor device according to the invention is designed to be placed up against the heart, it is made using entirely biocompatible materials and all its components have rounded and/or bevelled edges to avoid any injury to the cardiac muscle even in the event of accidental contact.

The two jaws 1a,b are shaped so as to guarantee an effective juxtaposition with the profile of the sternum and are wide enough to contain the surface pressure exerted thereon. Small vertical holes are created in the supporting surfaces to make easier the fixing of the retractor device to the sternal stumps and thus avoid any displacement of the retractor that might potentially be caused by movements of the rib cage and/or of the patient.

The device will be made in different sizes to guarantee that its overall longitudinal dimensions do not exceed the length of the sternum, and its dimensions in the direction orthogonal to the sternal plane do not extend too far beyond the edge of the surgical site in order to facilitate as far as possible the application of the sterile cover. Retractor devices may consequently be designed for use in neonatal surgery, for children and for various adult body sizes.

The actuator fluid is fed and withdrawn at the bottom of the sleeve 3 comprising the actuator, remaining fixed in position so that the fluid delivery tube emerges longitudinally with respect to the sterile cover on the patient's abdomen.

The retractor device according to the present invention enables numerous advantages to be achieved by comparison with the currently-adopted solutions. In the first place, it enables a safe and efficient sternum divarication to be achieved with a relatively standardisable procedure. Moreover, it enables the divarication amplitude to be adjusted from the outside without any need to reopen the surgical site, thus avoiding the removal of the protective membrane at any possible need. Depending on the patient's specific clinical needs, the operator can therefore increase or reduce the distance between the sternal stumps simply by taking action on the external syringe (or other actuator fluid insertion/withdrawal means) without removing the membrane. This versatility consequently also enables action to be taken promptly to deal with any deterioration in the patient's hemodynamic situation; on the other hand, it also enables a patient to be gradually “weaned” off the expanded sternal closure.

The retractor device according to the invention is made so as to guarantee absolutely sterile conditions and preferably of non-colonisable material. It may also be made in a resterilisable version or in a disposable version.

As concerns the primary use of the device according to the present invention for temporarily divaricating the sternum, it will be clear to a person skilled in the art that the device has other potential uses in the field of plastic surgery, in undermined lesions where a loss of substance makes it necessary to maintain the surface margins of the lesion open to enable in-depth wound irrigation from the outside, while reducing the distance between the margins as the tissue progressively grows back. The system is likewise applicable in all those modem surgery settings in which soft tissues need to be divaricated to enable the in-depth irrigation of a wound.

Various modifications and alterations may be appreciated based on a review of this disclosure. These changes and additions are intended to be within the scope and spirit of the invention as defined by the following claims.

What is claimed is:

1. A sternum retractor device suitable for temporarily maintaining a gap between sternal stumps during cardiac surgical procedures, the device comprising jaws configured so as to be inserted between the stumps and shaped for firmly engaging the stumps, an actuator connected to the jaws for controlling their mutual closing and opening movements, and a member for controlled feed or withdrawal of a selected biocompatible actuator fluid in/from the actuator, thereby enabling the achievement and maintenance of the gap required between the two sternal stumps as well as the progressive reduction of the gap as a function of a patient's condition.

2. The device set forth in claim 1, wherein the actuator operates in a direction substantially perpendicular to the direction of divarication and is connected to the jaws by an articulated mechanism that moves the jaws closer together or further away from one another as a result of such operation.

3. The device set forth in claim 2, wherein the actuator comprises a stationary part connected to the member for inserting/withdrawing the actuator fluid, and a movable part slidingly engaged inside the stationary part with a watertight seal, the articulated mechanism comprising a first and a second pair of arms pivotally connected to the stationary part and the movable part, respectively, the arms of the first and second pairs being revolvingly connected, two by two, to the jaws, each pair on a respective common pin.

4. The device set forth in claim 3, wherein the arms of the first pair extend from the end of a bar attached crosswise to the end of the stationary part opposite the end from which the movable part emerges.

5. The device set forth in claim 3, wherein the arms of the second pair are hinged onto a bracket extending along the movable part and at a distance therefrom that is generally greater than the thickness of the wall of the stationary part.

6. The device set forth in claim 2, wherein the stationary part is a tubular sleeve that is closed at one end and the movable part is a piston with a flat oval cross-section slidingly contained inside the sleeve with a watertight seal.

7. The device set forth in claim 2, wherein ports for the insertion/withdrawal of the actuator fluid are provided at the end of the fixed part opposite the end from which the movable part emerges.

8. The device set forth in claim 1, wherein the actuator operates substantially in the same direction as that of divarication and comprises a plurality of tubular sleeves connected telescopically and with a watertight seal, the jaws being fixed to the two terminal telescopic sleeves, the cross-section of the sleeves being a flat oval.

9. The device set forth in claim 1, wherein the means for inserting/withdrawing the actuator fluid in or from the actuator comprises a syringe for its insertion and a shut-off valve for its withdrawal, both operated manually.

10. The device set forth in claim 1, wherein the means for inserting/withdrawing the actuator fluid in or from the actuator comprises a dosing pump and a solenoid valve operated by a microcontroller.

11. The device set forth in claim 1, wherein the jaws have a substantially U-shaped cross-section.

12. The device set forth in claim 1, wherein fixing holes are provided along the jaws.

13. The device set forth in claim 1, wherein the jaws, the actuator and the articulated mechanism have rounded or bevelled edges.

14. The device set forth in claim 1, wherein the actuator fluid is a selected biocompatible liquid.
